(19) United States
(12) Patent Application Publication
(10) Pub. No.: US 2007/0072828 A1
(43) Pub. Date: Mar. 29, 2007

(54) Bile preparations for colorectal disorders

(76) Inventor: Seo Hong Yoo, Wyckoff, NJ (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/522,162

(22) Filed: Sep. 15, 2006

Related U.S. Application Data
(63) Continuation-in-part of application No. 09/778,154, filed on Feb. 5, 2001.
(63) Continuation-in-part of application No. 09/357,549, filed on Jul. 20, 1999, now Pat. No. 6,251,428.
(63) Continuation-in-part of application No. 10/996,945, filed on Nov. 24, 2004.
Application No. 10/996,945 is a continuation-in-part of application No. 09/778,154, filed on Feb. 5, 2001.
Application No. 10/996,945 is a continuation-in-part of application No. 09/357,549, filed on Jul. 20, 1999, now Pat. No. 6,251,428.
(60) Provisional application No. 60/180,268, filed on Feb. 4, 2000.
(60) Provisional application No. 60/094,069, filed on Jul. 24, 1998.
(60) Provisional application No. 60/180,268, filed on Feb. 4, 2000.
(60) Provisional application No. 60/094,069, filed on Jul. 24, 1998.

(51) Int. Cl. A61K 31/718; A61K 31/56

(52) U.S. Cl. 514/60; 514/169; 514/171

(57) ABSTRACT

The present disclosure relates to methods and compositions to ameliorate or treat at least one symptom of colorectal cancer and/or adenomatous polyposis coli (APC). For example, some embodiments of the methods and compositions may reduce recurrence of colorectal adenomas and/or extend the life of a subject having colorectal cancer and/or APC. Some embodiments of the disclosure include maintaining a the total body weight in a subject having colorectal cancer and/or APC. According to some embodiments, a method of the disclosure may include administering a bile acid composition to a subject. A bile acid composition may include, in some embodiments, an aqueous solution that is free or substantially free of precipitates or particles. A aqueous solution may include (1) a bile acid, an aqueous soluble derivative of a bile acid, a bile acid salt, and/or 7-ketolithocholic acid, (2) a carbohydrate, and (3) water. An aqueous composition may further include an alkali.

### CROSS-REFERENCE TO RELATED APPLICATIONS

This application is a continuation-in-part of U.S. application Ser. No. 09/778,154 filed Feb. 5, 2001, which claims the benefit of U.S. Provisional Application No. 60/180,268 filed Feb. 4, 2000, and is a continuation-in-part of U.S. application Ser. No. 09/357,549 filed Jul. 20, 1999, now U.S. Pat. No. 6,251,428, which claims the benefit of U.S. Provisional Application No. 60/094,069 filed Jul. 24, 1998, each of which are incorporated herein, in their entirety, by reference. This application is also a continuation-in-part of U.S. application Ser. No. 10/996,945 filed Nov. 24, 2004, which is a continuation-in-part of U.S. application Ser. No. 09/778,154 filed Feb. 5, 2001, which claims the benefit of U.S. Provisional Application No. 60/180,268 filed Feb. 4, 2000, and is a continuation-in-part of U.S. application Ser. No. 09/357,549 filed Jul. 20, 1999, now U.S. Pat. No. 6,251,428, which claims the benefit of U.S. Provisional Application No. 60/094,069 filed Jul. 24, 1998, each of which are incorporated herein, in their entirety, by reference.

### TECHNICAL FIELD

The present disclosure is related to methods and compositions of one or more bile acids that may be used to ameliorate one or more symptoms of a colorectal disorder.

### BACKGROUND OF THE DISCLOSURE

Colorectal cancer (CRC) is one of the most common forms of gastrointestinal cancer in the world today and a leading cause of cancer-related deaths throughout the world. In the Asia-Pacific area, it is the fastest emerging gastrointestinal cancer. Colorectal cancer is the third most common cancer worldwide and the second leading cause of cancer deaths in men and women in the U.S. Cancers of the colon and rectum are rare in developing countries, but are the second most frequent malignancy in affluent societies. More than 940,000 cases occur annually worldwide and nearly 500,000 die from it each year. The American Cancer Society has estimated that there will be about 106,680 new cases of colon cancer and 41,930 new cases of rectal cancer in 2006 in the United States. Combined, they will cause about 55,170 deaths. It is a prevalent disease that is associated with considerable mortality and morbidity rates. More than 1,000,000 new cases and 500,000 deaths were expected, worldwide, in 2004.

The 5-year relative survival rate for people, whose colorectal cancer is treated in an early stage, before it has spread, is greater than 90%. But only 39% of colorectal cancers are found at that early stage. Once the cancer has spread to nearby organs or lymph nodes, the 5-year relative survival rate goes down, and if cancer has spread to distant organs (i.e., the liver or lung) the 5-year survival is less than 10%.

Prevention of cancerous lesions of CRC by endoscopic screening is promising, but costs are high and identification of high-risk populations is difficult. Screening both average-risk and high-risk populations for CRC may also have logistic and financial limitations.

### SUMMARY

Therefore, a need has arisen for methods and compositions that treat colorectal cancer and/or ameliorate at least one symptom of colorectal cancer.

The present disclosure relates to methods and compositions to ameliorate or treat at least one symptom of colorectal cancer and/or adenomatous polyposis coli (APC). For example, some embodiments of the methods and compositions may reduce recurrence of colorectal adenomas and/or extend the life of a subject having colorectal cancer and/or APC. Some embodiments of the disclosure include maintaining total body weight in a subject having colorectal cancer and/or APC. According to some embodiments, a method of the disclosure may include administering a bile acid composition to a subject. A bile acid composition may include, in some embodiments, an aqueous solution that is free or substantially free of precipitates or particles. A aqueous solution may include (1) a bile acid, an aqueous soluble derivative of a bile acid, a bile acid salt, and/or 7-ketolithocholic acid, (2) a carbohydrate selected from the group consisting of an aqueous soluble starch conversion product, an aqueous soluble dietary carbohydrate that escape digestion and absorption in the small intestine, and combinations thereof, and (3) water. An aqueous composition may further include an alkali.

According to some embodiments of the disclosure, a method of protecting a colorectum against adenomatous polyposis coli in a subject may include administering to the subject a composition comprising (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and water, wherein the first material and the carbohydrate both remain in solution for all pH values of the solution within a selected range of pH values (e.g., all pH values attainable in an aqueous system).

The disclosure also relates, in some embodiments, to a method of extending a subject's life including (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and (c) water, wherein the subject has or is at risk of having adenomatous polyposis coli and/or colorectal cancer and wherein the first material and the carbohydrate both remain in solution for all pH values of the solution within a selected range of pH values (e.g., all pH values attainable in an aqueous system).

A method of maintaining a total body weight in a subject having adenomatous polyposis coli and/or colorectal cancer, in some embodiments, may include administering to the subject a composition comprising (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and water, wherein the first material and the carbohydrate both remain in solution for all pH values of the solution within a selected range of pH values (e.g., all pH values attainable in an aqueous system).

In addition, according to some embodiments of the disclosure, a method of ameliorating or treating at least one symptom of adenomatous polyposis coli in a subject having or at risk of having adenomatous polyposis coli may include administering to the subject a composition comprising (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and water, wherein the first material and the carbohydrate both remain in solution for all pH values of the solution within a selected range of pH values (e.g., all pH values attainable in an aqueous system).

Some embodiments of the disclosure include a method of reducing recurrence of colorectal adenomas in a colorectum of a subject, which may include administering to the subject a composition comprising (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and water, wherein the first material and the carbohydrate both remain in solution for all pH values of the solution within a selected range of pH values (e.g., all pH values attainable in an aqueous system).

According to some embodiments, a method of ameliorating or treating at least one symptom of colorectal cancer in a subject having or at risk of having colorectal cancer may include administering to the subject a composition comprising (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble dietary carbohydrate that escapes digestion and absorption in the small intestine, and combinations thereof, and water, wherein the first material and the carbohydrate both remain in solution for all pH values of the solution within a selected range of pH values (e.g., all pH values attainable in an aqueous system).

A composition, according to some embodiments of the disclosure, may be clear (i.e., free or substantially free of precipitates or particles) and may include (a) a first material selected from the group consisting of a bile acid, an aqueous soluble derivative of a bile acid, a bile acid salt, and 7-ketolithocholic acid, (b) a carbohydrate selected from the group consisting of an aqueous soluble starch conversion product, an aqueous soluble digestion resistant maltodextrin (fibersol-2), and combinations thereof, and (c) water, wherein the first material and the carbohydrate both remain in solution for all pH values of the solution within a selected range of pH values. The weight ratio of aqueous soluble starch conversion product to aqueous soluble digestion resistant maltodextrin (fibersol-2) may be 0-100:100-0 (e.g., 1-99:99-1).

A composition, in some embodiments, may be clear (i.e., free or substantially free of precipitates or particles) and may include (a) a first material selected from the group consisting of a bile acid, an aqueous soluble derivative of a bile acid, a bile acid salt, and a bile acid conjugated with an amine by an amide linkage, (b) a second material comprising a high molecular weight aqueous soluble starch conversion product, and (c) water, wherein the first and second materials both remain in solution for all pH values of the solution within a selected range of pH values.

According to some embodiments of the disclosure, a composition may be administered to a subject in a liquid form (e.g., a clear solution, a syrup, a paste, a jelly, or a mucilage). According to some embodiments of the disclosure, a composition may be administered to a subject in a dried or solid form.

### BRIEF DESCRIPTION OF THE DRAWINGS

Some specific example embodiments of the disclosure may be understood by referring, in part, to the following description and the accompanying drawings, wherein:

FIG. 1A shows a representative mouse treated with AOM alone (left) with swollen testes and a bloody spot (arrow) and a representative mouse treated with AOM and fed Formulation 15 (right) with normal testes and no bloody spots.

FIG. 1B shows an enlarged image of the bloody spot (arrow) on the AOM-treated mouse shown in FIG. 1A.

FIG. 2 shows a representative mouse treated with AOM alone with blindness in the left eye and a tumor (arrows).

FIG. 3A shows a representative AOM-treated mouse with adenomatous polyps (arrows) on the colorectum as well as swollen testes and a bloody spot (arrow).

FIG. 3B shows a representative mouse treated with AOM and fed Formulation 15 without adenomatous polyps on the colorectum, with normal testes, and without bloody spots.

FIG. 4A shows a representative AOM-treated mouse with a tumor (arrow) on its back.

FIG. 4B shows a representative mouse treated with AOM and fed Formulation 15 with no tumors on its back.

FIG. 5A shows the tail of a representative AOM-treated Balb/c mouse with bloody spots (arrows).

FIG. 5B shows the tail of a representative Balb/c mouse treated with AOM and fed Formulation 15 with no bloody spots.

FIG. 6 is a graph showing the survival times for AOM-treated mice that did or did not receive Formulation 15.

FIG. 7 is a graph showing the body weight for AOM-treated mice that did or did not receive Formulation 15.

FIG. 8A shows a section of resected intestine from a representative mouse treated with AOM alone (left) with adenomatous polyps (arrows) and a section of resected intestine from a representative mouse treated with AOM and fed Formulation 15 (right) with no adenomatous polyps.

FIG. 8B shows a section of a resected intestine from another representative mouse treated with AOM alone.

### DETAILED DESCRIPTION

Colorectal Cancer

While the etiology of colon cancer has not been fully defined, numerous factors may be implicated as having a role in the onset of the carcinogenesis process. For example, epidemiological, case control and cohort studies suggest that a relationship may exist between dietary fat and colon cancer. In addition, a positive correlation may exist between colon cancer incidence and the elevation of the levels of fecal bile acids. This is in accord with and may be explained, in part, by an observation that elevation of dietary fat intake may increase the excretion of secondary bile acids.

The pathogenesis of inflammatory bowel disease (IBD)-associated colorectal carcinogenesis may involve a step-wise progression from inflamed and hyperplastic epithelia through flat dysplasia to finally adenocarcinoma. IBD-associated colorectal carcinogenesis may be promoted by chronic inflammation. However, mucosal inflammation may result in colonic carcinogenesis through one or more mechanisms. Such mechanisms may include, for example induction of genetic mutations, increased-cryptal cell proliferation, changes in crypt cell metabolism, changes in bile acid enterohepatic circulation, alterations in bacteria flora, and/or combinations thereof. One or more of these events may promote IBD-associated colorectal cancer development.

Azoxymethane (AOM) is a pro-carcinogen, which may be used to induce colorectal cancer in experimental animals. Dextran sulfate sodium salt (DSS) is a pro-inflammatory irritant, which may erode intestinal-tract epithelial cells. This may allow entry of enteric bacteria and provoke an inflammatory response to fight the infection. For example, in male mice, a single intraperitoneal injection of AOM (10 mg/kg body weight) followed by 1-week exposure to 2% DSS in the drinking water efficiently produced colonic adenocarcinomas (100% incidence) with the presence of tubular adenoma, dysplasia, and colitis with mucosal ulceration. A 1-week treatment with 2% DSS after AOM exposure is enough to produce colonic adenocarcinomas, suggesting a powerful tumor-promoting ability of DSS in male ICR mouse colon.

Adenomatous polyposis coli (APC)-associated polyposis conditions include familial adenomatous polyposis (FAP), attenuated FAP, Gardner syndrome, and Turcot syndrome. Incidence may vary from 1 case in 6,850 persons to 1 case in 31,250 persons in USA. FAP is an autosomal dominant inherited disorder characterized by the presence of hundreds to thousands of adenomatous polyps throughout the colon. All patients with this syndrome develop colon cancer if they are not treated, beginning at a mean age of 16 years (range 7-36 years). By age 35 years, 95% of individuals with FAP have polyps; without colectomy, colon cancer is inevitable. The mean age of colon cancer diagnosis in untreated individuals is 39 years (range 34-43 years). Extra-colonic manifestations are variably present and include polyps of the gastric fundus and duodenum, osteomas, dental anomalies, congenital hypertrophy of the retinal pigment epithelium (CHRPE), soft tissue tumors, desmoid tumors, and associated cancers. Attenuated FAP is characterized by a significant risk for colon cancer, but fewer colonic polyps (average of 30) than classic FAP, more proximally located polyps, and diagnosis of colon cancer at a later age; management may be substantially different. Gardner syndrome is characterized by colonic polyposis typical of FAP together with osteomas and soft tissue tumors. Turcot syndrome is the association of colonic polyposis and CNS tumors; the phenotypic features of Gardner syndrome and Turcot syndrome relate to the location of the APC mutation and are generally expressed in families with FAP.

Secondary Bile Acids

Bile acids may be the most abundant end products of cholesterol metabolism. Primary bile acids, e.g., cholic acid (CA) and chenodeoxycholic acid (CDCA), may be synthesized in the liver and secreted into the bile. Primary bile acids may be reabsorbed in the ileum, but some percentage may pass into the colon. In the colon, anaerobic bacteria metabolize primary bile acids and secondary bile acids are formed. Secondary bile acids, including deoxycholic acid (DCA) and lithocholic acid (LCA), are found in both the aqueous and solid portions of stool.

Without being limited to any particular mechanism of action, a secondary bile acid, e.g., deoxycholic acid (DCA), may influence the pathogenesis of colorectal cancer. For example, a secondary bile acid may disrupt the balance between colorectal crypt cell proliferation, differentiation, and apoptosis. A secondary bile acid may modify intracellular signaling and/or gene expression. For example, DCA may influence one or more pathways that regulate the activity of activator protein-1.

Without being limited to any particular mechanism of action in any particular embodiment, bile acid feeding may potentiate shedding of surface epithelial cells and/or may stimulate the proliferative activity of colonic epithelium. Increased epithelial shedding may be due to detergent properties of bile acids. Increased cell proliferation may be a secondary response to the loss of surface epithelium and/or due to direct action of the bile acids on the metabolic activity of the colonic epithelium.

DCA and other secondary bile acids may be cytotoxic to colonic epithelial cells, may be mutagenically active in bacterial test systems, may be associated with dysplasia, and/or may have anti-apoptotic properties. A bile acid may modulate colonic tumor development in an azoxymethane (AOM) model of experimental carcinogenesis. Some references appear to implicate one or more secondary bile acids in colorectal cancer as co-carcinogens. Indeed, some people with colorectal cancer may excrete higher levels of secondary bile acids in their stool.

A subject on a diet high in fat may produce or accumulate more biliary secondary bile acids than a subject on a diet with less fat. High concentrations of secondary bile acids, in particular deoxycholic acid (DCA), may damage colonic epithelium and/or accelerate carcinogenesis. Colonic adenoma or polyp formation may precede colorectal cancer formation. Without restricting any embodiment to any particular mechanism of action, the concentration of DCA and other bile acids in the aqueous phase of stool may be of greater importance to colon carcinogenesis than those in the solid phase of stool because bile acids in the aqueous phase may be more likely to contact colonic epithelium.

According to some embodiments of the disclosure a dietary carbohydrate may include carbohydrates that escape digestion and absorption in the small intestine. For example, a dietary carbohydrate may include non-digestible oligosaccharides (carbohydrates with a degree of polymerization between three and ten), resistant starch and non-starch polysaccharides.

Dietary fiber may include non-starch polysaccharides such as cellulose, hemicellulose (composed of a variety of heteropolysaccharides including arabinoxylans), β-glucan, and pectins. Resistant starch is another component of plant foods that may escape absorption and digestion in the small intestine and may behave, at least physiologically, like dietary fiber. Non-digestible oligosaccharides may stimulate growth of potentially beneficial bacteria such as Bifidobacteria in the gut.

Fecal concentrations of total and secondary bile acids were significantly lower following dietary carbohydrate feeding. Thus, dietary carbohydrate feeding may inhibit the conversion of pirmary bile acids to secondary bile acids. By contrast, concentrations of primary bile acids were unaffected by dietary carbohydrates feeding.

Sprague Dawley rats on 4-week diets including sucrose or resistant starch may be compared. Total bile acids in feces have been observed to be significantly lower in rats fed a resistant starch diet than in rats fed a sucrose diet. This may be due, at least in part, to reduced levels of deoxycholic, alpha-muricholic and beta-muricholic acids. In addition, fecal and cecal pH values have been observed to be significantly lower in rats fed a resistant starch diet than in rats fed a sucrose diet. Colon mucosal proliferation has been observed to be significantly lower in rats fed a resistant starch diet than in rats fed a sucrose diet. The reduction was apparent in the middle and upper compartments of the colon crypts. For example, mucosal proliferation, expressed as percentage of labeled cells in the upper compartment of the colon crypts, was positively correlated with the concentration of deoxycholic, lithocholic, alpha-muricholic, beta-muricholic, cholic and total bile acids in the feces. Resistant starch consumption may decrease colonic mucosal proliferation as a result of the decreased formation of cytotoxic secondary bile acids. Decreased formation of cytotoxic secondary bile acids may be mediated through acidification of the large bowel by production of short-chain fatty acids.

Non-Steroidal Anti-Inflammatory Drugs (NSAIDs)

Colorectal carcinogenesis is a multistep process including, for example, molecular and cellular alterations that may result in an identifiable precursor lesion, i.e., an adenomatous polyp. The transition from normal mucosa to adenoma and its subsequent progression to carcinoma may be protracted events that offer opportunities for preventive intervention(s). Non-steroidal anti-inflammatory drugs (NSAIDs) including, e.g., nimesulide, inhibited both colon tumorigenesis and colitis. Without limiting any embodiment, a correlation between increased cyclooxygenase-2 (COX-2) expression and colonic carcinoma and/or inflammation may suggest that one or more chemopreventive effects of NSAIDs may be mediated, at least in part, by COX inhibition. Accordingly, one or more NSAIDs and selective COX-2 inhibitors may reduce the incidence and mortality of CRC.

However, long-term use of NSAIDs is associated with substantial gastrointestinal toxicity and may cause an exacerbation in IBD patients. NSAID-related gastrointestinal injury remains a serious and clinically important issue. Gastrointestinal side effects associated with NSAID use are common. NSAID-associated dyspepsia occurs in up to 50% of patients who use these drugs, and heartburn, nausea, vomiting, and abdominal pain can also be observed. More important, however, is the link between NSAID use, gastrointestinal mucosal injury, and associated complications. Up to 100% of patients taking nonselective NSAIDs may demonstrate subepithelial hemorrhage, about 50% may have erosions (small, shallow breaks in the gastrointestinal mucosa), and 20% or more may have ulceration (injury extending through the muscular mucosa).

Regular NSAID use may reduce the incidence of colorectal cancer (CRC) in humans by 30% to 50%. NSAIDs may also have one or more chemotherapeutic properties alone and/or in combination with conventional treatment strategies. For example, sulindac may result in polyp regression in FAP patients. Traditional NSAIDs (e.g., aspirin, nimesulide and sulindac) may exert a portion of their chemopreventive actions through the inhibition of cycloxygenases (COXs), key enzymes in prostaglandin (PG) biosynthesis. There may be at least two isoforms of COX, COX-1 and COX-2. COX-1 may be constitutively expressed in most tissues and may have a role in normal tissue homoeostasis, whereas COX-2 may not be detectable in a majority of normal tissues, but may be induced by one or more cytokines and/or mitogens. COX-2 also may be elevated in 80% to 90% of CRCs, as well as a significant subset of adenomas (depending on their size). Inhibition of COX-1 by traditional NSAIDs may be responsible for side effects such as gastric bleeding, whereas inhibition of COX-2 may account for a chemopreventive activity of an NSAID. Administering a COX-2 selective NSAID (e.g., rofecoxib and celecoxib) may reduce the incidence and/or intensity of upper gastrointestinal disease associated with COX-1 inhibition. In addition, COX-2-selective NSAIDs may have many of the anti-neoplastic properties of traditional NSAIDs. For example, the COX-2-selective inhibitor celecoxib reduces the number, size and overall colorectal polyp burden in FAP patients.

Ursodeoxycholic Acid (UDCA)

Ursodeoxycholic acid (UDCA), a tertiary bile acid, may have one or more biological activities that differ from DCA. In some embodiments, a biological activity of UDCA may be diametrically opposed to a biological activity of DCA. For example, orally administering UDCA may reduce the amount of DCA in the aqueous-phase stool. UDCA may also suppress one or more pathways that DCA may activate, e.g., a mitogen-activated protein kinase pathway. In addition, UDCA may ameliorate, block, or prevent colon carcinogenesis and/or vital cell proliferation signal transduction pathways.

For example, in a 32 week rat study, UDCA at a daily dose of 240 mg/kg (1,440 mg/m², which is 2.6 times the maximum recommended human dose based on body surface area) suppressed the colonic carcinogenic effect of AOM. UDCA treatment also may be associated with (1) a decrease in the incidence of colorectal neoplasia in patients with primary biliary cirrhosis, (2) a reduced prevalence of colorectal neoplasia in patients with primary sclerosing cholangitis, and (3) eradication of severe colonic mucosal dysplasia in patients with ulcerative colitis and primary sclerosing cholangitis (PSC). In some embodiments, the chemoprotective effects of UDCA may begin to emerge months (e.g., at least one month) or years (e.g., at least one year) after administration is initiated. For example, consumption of a daily UDCA dose of 13-15 mg/kg of body weight for as long as 12 years was observed to be associated with a statistically significant reduction in colorectal neoplasia in patients with ulcerative colitis and primary sclerosing cholangitis. In this case, chemopreventive effects of UDCA began to emerge only after 6 years of the intervention.

In one study, administration of UDCA was observed to reduce the overall rate of recurrence of sporadic colorectal adenomas, but it is unclear whether the difference was statistically significant. It is possible that a longer observation period and/or a higher UDCA dosage may have revealed even greater efficacy. In this same study, a statistically significant UDCA-related reduction in recurrence of adenomas with high-grade dysplasia was observed.

UDCA treatment decreased tumors throughout the entire intestine in a dose-dependent fashion, compared with control treatment in an APC-mutant Min mouse model for familial polyposis coli. Combined treatment with UDCA plus sulindac, which may be active in the treatment of familial polyposis coli, was more effective than either agent alone for the prevention of tumors throughout the entire intestine. Thus, UDCA may be a useful agent to manage patients with this rare genetic disorder. There was no difference in weight gain, but there was some mortality among mice treated with ursodeoxycholic acid as a single agent (⅞ survived at 500 ppm, and 6/8 survived at 1500 ppm). In the group treated with a combination of ursodeoxycholic acid plus sulindac, 8 of 8 mice survived, and 7 of 8 survived when treated with 1500 ppm ursodeoxycholate combined with sulindac. A dose of about 50 to 7500 mg per day of UDCA with sulindac (NSAIDs) may protect a colorectum against a recurrence of colorectal adenomas.

UDCA may be a major component of bear bile. UDCA may be useful as a pharmaceutical agent for the treatment of and/or protection against one or more types of liver disease. In some embodiments of the disclosure, a bile acid (e.g., UDCA) may be used for dissolution of radiolucent gall stones and/or treating a cholestatic disorder selected from the group consisting of primary biliary cirrhosis, primary sclerosing cholangitis, intrahepatic cholestasis of pregnancy, cystic fibrosis-associated liver disease, a number of pediatric liver disorders, and chronic graft-versus-host disease of the liver.

A pharmacological action of UDCA may include dose-dependent (1) replacement and/or displacement of toxic bile acids with UDCA, (2) cytoprotective effects, (3) stabilization/protection of cell membranes, (4) anti-apoptotic effects, (5) immunomodulatory effects due to activation of the intracellular glucocorticoid receptor, (6) anti-inflammatory effects due to repression of NF-kB and inhibition of the induction of nitric oxide synthase, (7) stimulation of bile secretion, (8) stimulation of exocytosis, (8) insertion of canalicular membrane transporters, and (10) antioxidant action.

Following oral administration, approximately 30 to 60% of UDCA may be absorbed along the length of the jejunum and ileum by nonionic passive diffusion. UDCA may be absorbed in the ileum by active transport mechanisms and to a small extent (e.g., 20% of an ingested dose) in the colon. In healthy volunteers given 500 mg UDCA with ¹⁴C tracer, 30-44% of the dose was excreted as solid UDCA in feces due to extremely slow and incomplete dissolution and insolubility.

Once taken up by hepatocytes, UDCA may be conjugated to, for example, tauro-conjugated UDCA (TUDCA) and GUDCA and/or excreted in bile by hepatic first-pass clearance. Consequently, UDCA blood levels may be extremely low in the systemic circulation. Bile acids may undergo extensive hepatic recycling, or free UDCA may be secreted by hepatocytes in bile. UDCA secreted in bile may be actively and efficiently reabsorbed by cholangiocytes. UDCA and GUDCA are absorbed by both active and passive transport mechanisms, while TUDCA may be transported actively in the terminal ileum.

In some embodiments of the disclosure, a formulation is administered to treat and/or eradicate Helicobacter pylori infection and hepatitis C virus infection. In some embodiments of the disclosure, UDCA is administered to treat acute or chronic inflammatory diseases of liver and intestine efficiently. Non-limiting examples of inflammatory diseases include PBC, PSC and bowel disease. In some embodiments, the administration of UDCA significantly decreased hepatocarcinogenesis. In some embodiments of the disclosure, UDCA is administered to treat hypercholesterolemia.

In some embodiments, UDCA is practically insoluble in pH 1 to pH 8 and incompletely absorbed by nonsaturated passive absorption in the upper gastrointestinal tract, and actively in the distal ileum. It is partially metabolized by intestinal bacteria to lithocholic acid, which it enterocycled to the liver, undergoes sulfation and conjugation and is mainly excreted via the kidneys. Absorbed UDCA undergoes hepatic conjugation with glycine and taurine, and then undergoes enterohepatic cycling. There is a very high efficient and constant extraction by the hepatocytes of enterohepatic cycling bile acids.

In some embodiments, the pH in the digestive tract including stomach and colon may not be favorable to the solubilization of UDCA. The very slow solubilization at duodenum-jejunum-ileum may bring about an extended absorption over the time course which may be reflected in extremely low plasma levels of UDCA. In general, for a soluble drug in the stomach and jejunum its Tmax and Cmax in plasma depend on the passive jejunal absorption. Therefore, in the case of UDCA, which may be poorly soluble in the gastro-intestinal content, early duodenal absorption of solubilized UDCA may be followed by further ileal absorption of the progressively solubilized solid form of the UDCA.

In some embodiments, UDCA may never be entirely solubilized in the jejunal content, a variable proportion of the drug being in solid form. This proportion is not related to the initial amount of UDCA ingested. Solubilization of UDCA in the gastro-jejunal juice may be dependent on physiological conditions including, for example, pH in fasting state. UDCA may be practically insoluble in acidic pH. indeed, UDCA may be precipitated in a weak alkaline environment (up to pH 8).

In some embodiments, UDCA may be in solution and directly contact colonic epithelium to protect a colorectum against a recurrence of adenomas. Thus, the portion of fecal bile acids that is not in solution may have little therapeutic significance. Accordingly, it may be more important to determine the active water-soluble fraction of fecal bile acids than the total bile acids present in the feces, most of which are present in an inactive insoluble fecal solid state. Bile acids may act as intracellular signaling agents, which modulate cellular transport, alter intracellular Ca²⁺ levels, and/or activate cell surface receptors.

In some embodiments, the solubility of UDCA in aqueous solution may be from about 0.02 g per liter to about 60 g per liter. The solubility of UDCA, according to some embodiments, may be about 3,000 times higher than that of commercialized UDCA (e.g., 0.15 mol vs. 0.05 mmol). UDCA solubility may be about 300 times higher than that of TUDCA. Solubility of deoxycholic acid (DCA) may be 0.24 g per liter. DCA may be about 70 times more soluble than UDCA.

In some embodiments, a dosage form (e.g., oral, parenteral) may contain, for example, 500 mg of UDCA. A dosage form may have a Cmax that is more than about 8 times higher than an existing commercial UDCA form and/or a Tmax that is about 4-6 times faster than an existing commercial UDCA form. Fast Tmax and high Cmax mean that solubilized UDCA may be absorbed from the upper stomach very efficiently. Absorbed solubilized UDCA may cross the gastric mucosa.

In some embodiments, a dosage form (e.g., oral, parenteral) may contain, for example, 650 mg of UDCA. A dosage form may have a Cmax that is more than about 5 times higher Cmax than an existing commercial UDCA form and/or a Tmax that is about 1.4 times faster than an existing commercial UDCA form.

In some embodiments, the viscosity of a solution of the disclosure may influence residual time in an organ (e.g., stomach, duodenum, jejunum, ileum, colon, rectum and/or blood) and may influence delivery of solubilized UDCA to an organ. Solubilized UDCA may cross a cell membrane of, for example, stomach, duodenum, jejunum, ileum, colon and rectum.

A solution of the disclosure may, in some embodiments, display significantly increased aqueous solubility of UDCA, increased membrane permeability, and protection from epimerization of UDCA to CDCA.

In some embodiments, a UDCA dose above 10-12 mg/kg per day may not further increase its proportion in bile. Without limiting any embodiment of the disclosure, where this occurs, it may be because (1) UDCA may be biotransformed to CDCA through 7-keto-lithocholic acid by intestinal bacteria and/or (2) UDCA may be converted to CDCA by epimerization of the 7β-hydroxyl group and further to lithocholic acid (LCA). Therefore, increasing doses of UDCA may decrease UDCA absorption.

In some embodiments, UDCA composition may include aqueous solubilized UDCA, which remains in solution at any pH. For example, a composition of the disclosure may be free or substantially free of precipitates or particles at any pH.

A composition of the disclosure may include a soluble starch and/or a soluble dietary carbohydrate. Without limiting any embodiment of the disclosure, the concurrent presence of UDCA, soluble starch, and soluble dietary carbohydrate in intestine may reduce or prevent biotransformation of UDCA to any of the hydrophobic bile acids that have been implicated in promoting colorectal cancer.

A composition of the disclosure may, in some embodiments, extend the resident time of solubilized UDCA in the stomach and/or permit UDCA to reach the entire gastrointestinal epithelium including the large intestine. A composition may, in some embodiments, include a dietary carbohydrate that escapes digestion and absorption in the small intestine selected from the group consisting of non-digestible oligosaccharides (carbohydrates with a degree of polymerization between three and ten), digestion resistant maltodextrin (fiber sol-2), guar gum, locust bean gum, and psyllium fibres) and non-starch polysaccharides. Administration of a composition that includes such a dietary carbohydrate may produce lower fecal concentrations of total and secondary bile acids.

In some embodiments of the disclosure, a composition may include a dietary carbohydrate. Non-limiting examples of dietary carbohydrate include guar gum, pectin, psyllium, oat gum, soybean fiber, oat bran, corn bran, cellulose, wheat bran and digestion resistant maltodextrin (fiber sol-2).

In some embodiments, a aqueous soluble UDCA salt may be administered orally; this UDCA salt may be simultaneously protonated and precipitated in acidic stomach. Precipitated UDCA may reach the colon through the intestine. Precipitated protonated UDCA may be insoluble (e.g., very insoluble) in the colon. Protonated UDCA may, in some embodiments, benefit from or require a relatively high pH for solubilization (e.g., pH=8.7) in order to protect a colorectum against a recurrence of adenomas efficiently.

In some embodiments, a bile acid composition of the disclosure may lack one or more of the disadvantageous features of existing commercial dosage forms of UDCA. In addition, a bile acid composition of the disclosure may, in some embodiments, contact a colorectal lesion without any precipitation and may function as a locally acting drug at the colon. Bile acid dosage forms, according to some embodiments of the disclosure, may be suitable or adaptable for oral and/or parenteral administration. In some embodiments, a bile acid composition of the disclosure may include an intact molecule of UDCA, an aqueous soluble starch conversion product (e.g., a product resulting from hydrolysis of starch) and soluble dietary carbohydrates that escape digestion and absorption in the small intestine. A bile acid composition, according to some embodiments of the disclosure, may include solubilized bile acid in water and may remain in aqueous solution without precipitation at any pH.

In some embodiments, a bile acid composition may be free or substantially free of precipitates or particles (e.g., based on visual inspection and/or light scattering). A composition free of precipitates or particles may be prepared, for example, by adding a bile acid to a basic solution, mixing until dissolved, and adding an aqueous soluble starch conversion product. A basic solution may include any common base. Non-limiting examples of common bases include ammonia, sodium hydroxide, sodium carbonate, potassium hydroxide, potassium carbonate, calcium hydroxide, and calcium carbonate. The molar ratio of base to bile acid may be from about 0.5 to about 20, from about 0.5 to about 15, from about 0.5 to about 10, from about 0.5 to about 5, from about 0.5 to about 1.5, from about 0.6 to about 1.3, and/or from about 1.0 to about 1.3.

In some embodiments, a method may include contacting a lesion caused by NSAIDS, alcohol and/or gastric irritating drug with a pharmaceutically effective amount of solubilized UDCA. A contacted lesion may be in the gastrointestinal tract and/or in the systemic circulation. A method may further include administering a composition of the disclosure to a subject.

Non-steroidal anti-inflammatory drugs (NSAIDs), which are drugs with analgesic, antipyretic and/or anti-inflammatory effects, may be associated with gastrointestinal toxicity mainly in the form of gastrointestinal mucosal damage like acute hemorrhagic erosions. In addition, the small bowel and/or colon may also be injured.

The present disclosure relates to an aqueous solution comprising (i) one or more soluble bile acids, aqueous soluble bile acid derivatives, bile acid salts, (ii) water, and (iii) one or more aqueous soluble starch conversion products and aqueous soluble dietary carbohydrate in an amount sufficient to produce a solution which does not form a precipitate at any pH within a desired pH range. The composition may contain a bile acid or a bile acid salt which itself has pharmaceutical effectiveness. Alternatively, according to some embodiments of the disclosure, the composition may comprise a non-bile acid pharmaceutical that is incompletely soluble.

In some embodiments, it may be an advantage of this disclosure that the bile acid and the carbohydrate remain in solution without precipitation at any pH from acidic to alkaline. These aqueous solution systems of bile acid are free or substantially free of precipitate or particles. A further advantage of some embodiments of this disclosure may be that the aqueous solution systems demonstrate no changes in physical appearance such as changes in clarity, color or odor following the addition of strong acids or alkali even after several months (e.g., 1, 2, 3, 4, 5, 6, 7, 8, 9, 10, 11, or 12 months) observation under accelerated conditions of storage at 50° C.

In some embodiments of the disclosure, an aqueous solution system of a bile acid may be administered orally and reach the gastrointestinal track without precipitation of a bile acid by exposure to acidic gastric juices and alkaline juices of the gastrointestinal track. These solubilized bile acid formulations may be effectively absorbed by direct contact between solubilized bile acid and intestine. Upon absorption, solubilized bile acid may undergo enterohepatic cycling. One or more bile acids may be dissolved in these aqueous solution systems as a therapeutically active agent, as an adjuvant of a drug, as a carrier of a drug or as an enhancer of drug solubility. A composition of the disclosure may be prepared, in some embodiments, for oral consumption, mouthwashes, gargles, nasal preparations, otic preparations, injections, douches, enemas, topical skin preparations, other topical preparations, and cosmetic preparations.

Soluble bile acids are any type of aqueous soluble bile acids. A bile acid salt is any aqueous soluble salt of a bile acid. Bile salts exhibit greater solubilizing capacity for phospholipid and cholesterol and are consequently better detergents. More hydrophobic bile salts may be more injurious to various membranes, both in vivo and in vitro. Aqueous dissolved salts of bile acids may be formed by the reaction of a (1) bile acid and (2) an alkali or an amine including but not limited to aliphatic free amines such as trientine, diethylene triamine, tetraethylene pentamine, and basic amino acids such as arginine, lysine, ornithine, and ammonia, and amino sugars such as D-glucamine, N-alkylglucamines, and quaternary ammonium derivatives such as choline, heterocyclic amines such as piperazine, N-alkylpiperazine, piperidine, N-alkylpiperidine, morpholine, N-alkylmorphline, pyrrolidine, triethanolamine, and trimethanolamine. According to some embodiments of the disclosure, soluble bile acid salts may also include aqueous soluble metal salts of bile acids, bile acid/cyclodextrin inclusion compounds, and aqueous soluble O-sulfonated bile acids. Soluble bile acid derivatives, according to some embodiments of this disclosure, may be those derivatives which are as soluble in aqueous solution as or more soluble in aqueous solution than is the corresponding underivatized bile acid. Bile acid derivatives include, but are not limited to derivatives formed at the hydroxyl and carboxylic acid groups of the bile acid with other functional groups including but not limited to halogens and amino groups. Soluble bile acid may include an aqueous preparation of a free acid form of bile acid combined with one of HCl, phosphoric acid, citric acid, acetic acid, ammonia, or arginine. Bile acids that may be used in accordance with the teachings of this disclosure include, without limitation, ursodeoxycholic acid, hyodeoxycholic acid, 7-oxolithocholic acid, and an aqueous soluble derivative of a bile acid.

In some embodiments, administration of a composition of the disclosure may result in higher in vivo levels of bile acids than at least some existing commercial preparations. Therefore, the therapeutic potential of bile acid may be more fully achieved than other formulations. In vivo levels of bile acids attainable with existing formulations in which bile is incompletely solubilized may be lower and require administration of larger amounts of bile acids. By contrast, in vivo levels of bile acids attainable with a composition according to an embodiment of the disclosure in which bile acid is completely or substantially completely dissolved, may be higher even where the dosage is the same or even lower.

In some embodiments of the disclosure, a plurality of bile acids may be used in a single formulation. Mixtures of two or more bile salts of differing hydrophobic activity may behave as a single bile salt of an intermediate hydrophobic activity. As a result, detergent properties and the toxicity of mixtures of two bile acids of differing hydrophobic activity often are intermediate between the individual components.

Mixtures of two or more bile salts of differing hydrophobic activity may behave as a single bile salt of an intermediate hydrophobic activity. As a result, detergent properties and the toxicity of mixtures of two bile acids of differing hydrophobic activity often are intermediate between the individual components.

Carbohydrates suitable for use in the disclosure include aqueous soluble starch conversion products and soluble dietary carbohydrates that escape digestion and absorption in the small intestine. According to some embodiments of the present disclosure, aqueous soluble starch conversion products include carbohydrates obtained (e.g., obtained directly) from the partial or incomplete hydrolysis of starch under various pH conditions. Non-limiting examples include maltodextrin, dextrin, liquid glucose, corn syrup solid (dried powder of liquid glucose), and soluble starch. In some embodiments, for example, MALTRIN®040 (D.E. 4-7), MALTRIN®050 (D.E. 4-7), MALTRIN® 070 (D.E. 6-9), MALTRIN®0100 (D.E. 9-12), MALTRIN ® 150 (D.E. 13-17), MALTRIN®180 (D.E. 16.5-19.5), MALTRIN® 200 (D.E. 20-23), MALTRIN® 250 (D.E. 23-27), MALTRIN®440 (D.E. 4-7), MALTRIN® 500 (D.E. 9-12), MALTRIN®550 (D.E. 13-17), MALTRIN®580 (D.E. 4-7), MALTRIN® M585 (D.E. 16.5-19.5), MALTRIN®600 (D.E. 20-23), MALTRIN®510 (D.E. 9-12), MALTRIN®520 (D.E. 9-12), and MALTRIN® M700 (D.E. 9-12) manufactured by GPC®, Grain Processing Corporation of Muscatine, Iowa may be used as maltodextrin, liquid glucose, and corn syrup solid (dried powder of liquid glucose). For the purpose of this embodiment, the term “corn syrup solid” is a dried form of liquid glucose. A starch conversion product may, in some embodiments, include at least one reducing end and/or at least one non-reducing end. If a starch conversion product is polymeric, it may be linear or branched. The molecular weight may be from about 100 mass units to over 106 mass units. High molecular weight aqueous soluble starch conversion products are those having a molecular weight over 105 mass units.

In some embodiments, cyclodextrin, the formation of which involves a condensation step that eliminates its free ends, may be regarded as neither an aqueous soluble starch conversion product nor an aqueous soluble non-starch polysaccharide. In some embodiments, a composition of the disclosure may be substantially free of cyclodextrin. In some embodiments, a composition of the disclosure may be completely free of cyclodextrin. Alternatively, in some embodiments of the disclosure, a formulation a composition of the disclosure may comprise cyclodextrin in addition to a starch conversion product and/or a non-starch polysaccharide.

The amount of high molecular weight aqueous soluble starch conversion product and/or soluble dietary carbohydrates that escape digestion and absorption in the small intestine used in embodiments of the disclosure is at least the amount needed to render a chosen bile acid(s) in the preparation soluble at the desired concentration and/or pH or pH range. The ratio of aqueous soluble starch conversion product to soluble dietary carbohydrates (soluble resistant starch) is from about 10-100 to about 90-0 by weight.

In some embodiments of the disclosure, the approximate minimal weight ratio of maltodextrin to UDCA required to prevent UDCA precipitation may be 6:1 (i.e. 1.2 g for every 0.2 g of UDCA, 6 g for every 1 g of UDCA, and 12 g for every 2 g of UDCA in 100 mL of water). In some embodiments of the disclosure, the approximate minimal quantity of maltodextrin may be 30 g for every 200 mg of chenodeoxycholic acid, 12 g for every 200 mg of 7-ketolithocholic acid, 10 g for every 200 mg of cholic acid and 50 g for every 200 mg of deoxycholic acid. In some embodiments of the disclosure, the approximate minimal weight ratio of liquid glucose (commercial light corn syrup) to UDCA required to prevent the precipitation of bile acids from the aqueous solution dosage forms of the disclosure may be about 25:1 (i.e. 12.5 g for every 500 mg UDCA in 100 mL water and 25 g for every 1 g ursodeoxycholic acid in 200 mL water). In some embodiments of the disclosure, the approximate minimal quantity of dried powder of liquid glucose (corn syrup solid, e.g. MALTRIN® M200) required to prevent the precipitation of bile acids from the aqueous solution dosage forms of the disclosure may be 30 g for every 1 g ursodeoxycholic acid in 100 mL water, and approximately 60 g for every 2 g of ursodeoxycholic acid in 200 mL water. In some embodiments of the disclosure, the approximate minimal quantity of soluble non-starch polysaccharide required to prevent the precipitation of bile acids from the aqueous solution dosage forms of the disclosure may be 50 g guar gum for every 500 mg ursodeoxycholic acid in 100 mL water and 80 g of pectin for every 500 mg of ursodeoxycholic acid in 100 mL water. The minimal required quantity of high molecular weight aqueous soluble starch conversion products and/or soluble non-starch polysaccharide, according to some embodiments, may be primarily determined by the absolute quantity of bile acids in the solution formulation rather than the concentration.

In some embodiments of the disclosure, a formulation may comprise a starch conversion product and soluble dietary carbohydrates that escape digestion and absorption in the small intestine with additional cyclodextrin (as taste masking).

In some embodiments of the disclosure, the formulation further comprises emulsifying agents. For the purpose of the disclosure, the term “emulsifying agent” includes emulsifying agents and suspending agents. Non-limiting examples of emulsifying agents include guar gum, pectin, acacia, carrageenan, carboxymethyl cellulose sodium, hydroxymethyl cellulose, hydroxypropyl cellulose, methyl cellulose, polyvinyl alcohol, povidone, tragacanth gum, xanthan gum, and Sorbian ester.

The selected pH range for which the formulation will not precipitate its bile acid, starch conversion product, soluble dietary carbohydrate that escapes digestion and absorption in the small intestine may be any range of pH levels obtainable with an aqueous system. In some embodiments, the range maybe between about pH 1 and about pH 14 and/or between about pH 1 and about pH 10. In some embodiments, a pH range may be any subset of the range of pH levels obtainable in an aqueous system sufficient for a pharmaceutical formulation to remain in solution from preparation, to administration, to absorption in the body, according to the method of administration. Thus, the composition may be used as a pharmaceutical formulation wherein a pharmaceutical compound and/or a bile acid remain in solution without precipitation at prevailing pH levels in the mouth, stomach and intestines.

Some embodiments of the disclosure, a solution may be practiced with pH adjustable agents. Non-limiting examples include HCl, H₃PO₄, H₂SO₄, HNO₃, CH₃COOH, citric acid, malic acid, tartaric acid, lactic acid, phosphate, eidetic acid and alkalis.

In some embodiments of the disclosure, a bile acid remains dissolved under acidic conditions as a free bile acid in spite of the general insolubility of bile acids under acidic conditions.

A solution, according to some embodiments, may be administered with one or more pharmaceutical compounds (e.g., a pharmaceutical compound may include analgesic, antipyretics, anti-inflammatory drugs, immunoactive drugs, antineoplastic drugs, antibiotics, anti-tumor agents). Administration of a bile composition of the disclosure with pharmaceutical compound may, in some embodiments, (a) increase the intensity of a response to the pharmaceutical compound, (b) increase the efficacy of the pharmaceutical compound, (c) decrease the required dose of the pharmaceutical compound, and/or (d) decrease the toxicity of the pharmaceutical compound. Solutions of the disclosure may also be administered separately, in terms of both the route and time of administration. Non-limiting examples of pharmaceutical compounds include aspirin, methyl salicylate, diflunisal, indomethacin, sulindac, diclofenac, ibuprofen, ketoprofen, naproxen, ketorolac, mefenamic acid, piroxicam, meloxicam, coxibscelecoxib rofecoxib, valdecoxib, parecoxib, etoricoxib, nimesulide. oxaliplatin, leucovorin, irinotecan, cimetidine, salicylic acid, 2, 2-Bis (4-(4-amino-3-hydroxyphenoxy) phenyl) adamantane (DPA), paclitaxel, oxaliplatin, 5-fluorouracil, azathioprine, mycophenolate mofetil, cyclosporine, mycophenolic acid, tacrolimus, sirolimus, basiliximab, daclizumab, anti-thymocyte globulin (Rabbit), allopurinol, palonosetron, dolasetron, pamidronate, rasburicase, aprepitant, amifostine, gefitinib, palifermin, granisetron, sargramostim, levothyroxine, dronabinol, pegfilgrastim, interleukin eleven, filgrastim, octreotide, cinacalcet, levothyroxine, Liotrix, dexrazoxane, ondansetron, zoledronic acid, celecoxib, fenoprofen, benorylate, faislamine, amoxiprin, carprofen, flurbiprofen, loxoprofen, tiaprofenic acid, meclofenamic, ketorolac, oxaprozin, etodolac, nabumetone, mesalamine, balsalazide, bevacizumab, alemtuzumab, cetuximab, aldesleukin, ibritumomab tiuxetan, pemetrexed, tositumomab, gemcitabine, imatinib, trastuzumab, altretamine, topotecan, interferon alfa-2b, procarbazine, gemtuzumab ozogamicin, vinorelbine, mitoxantrone, denileukin diftitox, rituximab, erlotinib, bexarotene, arsenic trioxide, bortezomib, tretinoin, doxorubicin, dactinomycin, epirubicin, idarubicin, pentostatin, busulfan, temozolomide, melphalan, chlorambucil, mechlorethamine HC, clofarabine, cytarabine, cladribine, mercaptopurine, thioguanine, capecitabine, bicalutamide, flutamide, anastrozole, exemestane, Fulvestrant, letrozole, estramustine , leuprolide, triptorelin pamoate, histrelin, goserelin, porfimer, rotenone, thenoyltrifluoroacetone (TTFA), antimycin A, myxothiazol or oligomycin dexamethasone, methylprednisolone, hydrocortisone, prednisolone, rotenone, thenoyltrifluoroacetone (TTFA), antimycin A, myxothiazol, oligomycin, valdecoxib, rofecoxib, parecoxib and etoricoxib, atorvastatin, cerivastatin, fluvastatin, lovastatin, pravastatin-sodium, simvastatin, rosuvastatin, mevastatin, pitavastatin.

In some embodiments of the disclosure, a formulation may be used to treat (e.g., ameliorate at least one symptom) human and mammalian diseases.

In some embodiments of the disclosure, a composition may be prepared and/or modified such that it may be administered as a liquid, solid, powder or, granular, capsule and tablet. In some embodiments of the disclosure, a composition may be comprised in a parenteral solution (e.g., an injectable solution, a solution, a syrup, a thick syrup or a paste), enemas, jellies and mucilages. A non-limiting example of a syrup is a solution of maltodextrin wherein the concentration of maltodextrin is less than 500 g/L. A non-limiting example of a syrup is a solution of maltodextrin wherein the concentration of maltodextrin is between 500 g/L and 1.0 kg/L inclusive. A non-limiting example of a thick syrup is a solution of maltodextrin wherein the concentration of maltodextrin is between 1.0 kg/L and 1.2 kg/L inclusive. A non-limiting example of a paste is a solution of maltodextrin wherein the concentration of maltodextrin is greater than 1.2 kg/L.

A jelly may be a class of gels in which a structural coherent matrix comprises a high portion of liquid, usually water. It is similar to mucilage, in that it may be prepared from similar gums, but it differs from the latter in having a jelly-like consistency. Non-limiting examples of a jelly include, for example, lidocaine hydrochloride jelly USP and therapeutic vaginal jellies. In some embodiments, a jelly may comprise a thickening agent (e.g., a water soluble polysaccharide and/or a synthetic cellulose derivative which swells in water). Non-limiting examples of a thickening agent for a jelly may include acacia, chondrus, gelatin, xanthan gum carboxymethylcellulose, hydroxyethylcellulose, hydroxypropylcellulose, and/or hydroxypropylmethylcellulose.

A mucilage may be a thick, viscid, adhesive liquid. A mucilige may be produced by dispersing or dissolving gum in water, or by extracting mucilaginous principles from vegetable substances with water. Non-limiting examples of a mucilage include, for example, acacia mucilage and tragacanth mucilage. Jellies and mucilages may be used primarily to cover the bitter taste of bile acid by minimizing physical contact of solubilized bile acid. In some embodiments, a mucilage may comprise a thickening agent (e.g., a water soluble polysaccharide and/or a synthetic cellulose derivative which swells in water). Non-limiting examples of a thickening agent for a jelly may include acacia, chondrus, gelatin, xanthan gum carboxymethylcellulose, hydroxyethylcellulose, hydroxypropylcellulose, and/or hydroxypropylmethylcellulose.

The stability of dosage formulations of the disclosure may be evaluated by measuring the concentration of the relevant bile acid over time in preparations comprising soluble bile acid, soluble carbohydrates (a high molecular weight aqueous soluble starch conversion product and dietary carbohydrate), and water at various pH and temperature levels. The retention time (high performance liquid chromatography) of each bile acid may be adjusted as needed to permit individual analysis each bile acid present in a complex sample, i.e., a sample having a plurality of bile acids. Stability tests may also be performed by assessing the light-scattering properties of a test solution. In addition, established accelerated testing conditions may be used.

In some embodiments, a composition of the disclosure may, without being filtered, remain substantially free of precipitates and particles for over one day, over two days, over three days, over one week, over two weeks, over three weeks, over four weeks, over five weeks, over six weeks, over seven weeks, over eight weeks, over nine weeks, over ten weeks, over eleven weeks, over twelve weeks, over fifteen weeks, over eighteen weeks, over twenty-one weeks, over twenty-four weeks, over nine months, over twelve months, over eighteen months, and/or over twenty-four months. In some embodiments, a composition of the disclosure may have greater than about 95% of the starting bile concentration, greater than about 96% of the starting bile concentration, greater than about 97% of the starting bile concentration, greater than about 98% of the starting bile concentration, and/or greater than about 99% of the starting bile concentration after one day, after two days, after three days, after one week, after two weeks, after three weeks, after four weeks, after five weeks, after six weeks, after seven weeks, after eight weeks, after nine weeks, after ten weeks, after eleven weeks, after twelve weeks, after fifteen weeks, after eighteen weeks, after twenty-one weeks, after twenty-four weeks, after nine months, after twelve months, after eighteen months, and/or after twenty-four months.

All stability tests performed on solutions of the disclosure were satisfactory in that the concentration of bile acid as measured by HPLC did not change appreciably over time at various pH levels. Particularly, all bile acid solution formulations tested showed excellent results in the stability tests with no precipitation and no physical appearance changes over the test period. Some formulations remain stable for over 2 years. The aqueous solution dosage forms according to this disclosure that were tested did not change either physically or chemically at various pH conditions under accelerated conditions despite the addition of therapeutically and chemically active agents that are stable and soluble in hydrochloric acid solution. Therefore, these aqueous solution systems may be useful pharmaceutical dosage forms for therapeutically active bile acids preparations, and/or drug (pharmaceutical compound) delivery preparations. In such preparations, a bile acid may play a role as a drug adjuvant, a drug, a carrier, or a drug solubility enhancer (e.g., by micelle formation) at various pH conditions without stability problems (e.g., including precipitation in acidic conditions).

Dried Forms of UDCA

According to some embodiments, a solution of the disclosure, may be dried or solid. For the purpose of this disclosure, a “primary” aqueous solution bile acid dosage formulation according to the disclosure is produced by the original combination of a bile acid or its salts and a carbohydrate with water (e.g., a parent solution). It may be prepared by a simultaneous or stepwise combination of ingredients. A “secondary” aqueous solution bile acid dosage formulation, by contrast, is a solution prepared from a powder or solid comprising previously co-dissolved bile acid and carbohydrate. Thus, a secondary aqueous solution bile acid dosage formulation differs at least in that water has been added, removed, and added again.

In some embodiments, a bile acid composition may be stored or administered in a dry and/or solid form. Thus, the present disclosure relates to dry or solid preparations of bile acids that form clear (i.e., free or substantially free of precipitates or particles) solutions (e.g., secondary solutions) upon exposure to water. Dry or solid forms of the disclosure may be prepared from clear (i.e., free or substantially free of precipitates or particles) solutions of bile acids (“parent solutions”). The present disclosure also relates to methods for preparing and/or solubilizing such dry and/or solid forms. These dry or solid formulations may have improved bioavailability, plasma bioavailability, and/or absorbability of a bile acid. Additionally, formulations of the disclosure may have improved bioavailability, plasma bioavailability and absorbability of one or more pharmaceutical compounds.

In some embodiments, a solution of the disclosure (e.g., a parent solution) may be dried or solid. For example, a dried or solid form may be prepared from a solution formulation of a bile acid by lyophilization and/or evaporation. A solution may be partially dried to produce a semi-solid form. The solutions may be thoroughly dried to form a solid, powder, and/or granule. Dried or solid forms of the aqueous solutions may be free or substantially free of water. Dried or solid forms may be dried by a fluid process, a tray process, a spray process, and/or a freezing process. Dried or solid forms may be administered directly, as solid dosage forms or combined with water prior to administration.

Compositions of the disclosure (e.g., dried or solid forms) may further comprise a disintegrant. A disintegrant may facilitate breakup or disintegration of a dry or solid form after administration. Disintegrants may include starches such as Veegum HV, methylcellulose, agar, bentonite, natural sponge, cation exchange resins, alginic acid, guar gum, citrus pulp, and carboxymethylcellulose, clays, celluloses, aligns, gums, and cross-linked polymers (crospovidone), cross-linked cellulose (Croscarmelose), and cross-linked starch (sodium starch glycolate). The disintegrating function may be due to capillary action rather than swelling. In general, the aqueous soluble disintegrants may be mixed with active ingredients prior to drying. For aqueous insoluble disintegrants, 5% starch by weight, may be added to the powder blends in the dry state. If more rapid disintegration is desired, this amount may be increased to 10 or 15%. Sodium starch glycolate at 2 to 4% swells 7-fold to 12-fold in less than 30 seconds and Croscarmelose swells 4-fold to 8-fold in less than 10 seconds.

Evolution of carbon dioxide may facilitate fast dissolution of dried or solid forms derived from the solution formulations of bile acid compositions. Dried or solid forms containing a mixture of sodium bicarbonate and an acidulant such as tartaric or citric acid will effervesce when added to water. The amount of sodium bicarbonate may be about ten times the amount of bile acid. The amount of acidulant may be twenty percent more than the amount of sodium bicarbonate. Sufficient acid is added to produce a neutral or slightly acidic reaction when dissolution in water is rapid and complete.

The disclosure further relates to the preparation of solution formulations derived from dried or solid forms of bile acid compositions. High throughput sonication with or without heating at about 60° C. may be useful in solubilizing dry or solid preparations of the disclosure. A high throughput sonication system may be used to drive precipitated compounds back into solution during preparation of solution formulations. The effects of sonication time, power, and amplitude may be optimized in order to drive compounds back into solution. Sonicator that generate sound energy at 20 kHz from 0-1150 watts may be used in forming clear aqueous solutions of the disclosure.

A dried or solid form, in some embodiments, may be prepared from a parent solution by wet granulation, dry granulation, and/or fluid-bed granulation. When ingredients have sufficient inherent binding or cohesive properties, dry granulation method (slugging) may be used to make granules. General steps of wet and dry granulation include weighing, mixing, granulation (slugging), and screening. Fluid bed granulation may be performed by spraying a granulating solution or solvent into or onto the bed of suspended particles, followed by rapid drying in suspending air. In these systems, suspended particles, which are dried forms derived from parent solutions, may be coated with granulating solution or solvent which contains enteric polymers. Enteric polymers may comprise cellulose acetate phthalate (CAP), which is capable of functioning effectively as an enteric coating at pH greater than 6, polyvinyl acetate phthalate (PVAP), methacrylic acid-methacylic acid ester copolymers, cellulose acetate trimellitate (CAT), carboxymethyl ethylcellulose (CMEC), and/or hydroxylpropyl methylcellulose acetate succinate (HPMCAS). This granulated form with those enteric polymers may remain intact in the stomach, but dissolve and release the active ingredient once it reaches the intestine and colon.

Spheronization, a form of pelletization, may include formation of spherical particles (spheres) from wet granulation or fluid bed granulation. Rod shaped cylindrical segments ranging in diameter from 500 microns to 12 millimeters may be prepared through an extruding machine. After extrusion the segments may be placed into the Marumerizer where they are shaped into spheres by centrifugal and frictional forces on a rotating plate. The pellets are dried and then coated. In some embodiments of the disclosure, dried or solid forms of the solution formulations of bile acid compositions may be prepared by a spheronization process and then coated with the enteric polymers.

In some embodiments of the disclosure, a primary aqueous solution bile acid dosage formulation may be dried by spray-drying. Spray-drying may include bringing together a highly dispersed liquid and a sufficient volume of hot air to produce evaporation and drying of the liquid droplets. The feed liquid may be a solution, slurry, syrup or paste provided it is pumpable and capable of being atomized. The liquid feed may be sprayed into a current of warm filtered air. The air may supply heat for evaporation and may convey the dried product to the collector; the air may then be exhausted with the moisture. Spray-dried powder particles may be homogeneous, approximately spherical in shape, nearly uniform in size, and frequently hollow. The hollow characteristic may result in low bulk density with a rapid rate of solution. This process may be useful in coating one material on another to protect the interior substance or to control the rate of its release. For example, a dried form of an aqueous solution bile acid dosage formulation may be coated with enteric polymers for the colonic delivery of solubilized UDCA. Dehydration may also be accomplished with lyophilization, evaporation or any other dehydration technique known in the art.

A dried form, (e.g. powder or solid), may be administered directly or recombined with water to produce a secondary clear aqueous solution bile acid dosage formulation. Secondary aqueous solution bile acid dosage formulations, i.e. those produced from dried forms, may have the same or substantially the same properties as primary formulations.

The disclosure contemplates the addition of additives such as pharmaceuticals to primary and secondary aqueous bile acid solutions as well as to dried or solid forms. If administered in dried or solid form, the material may be combined with one or more diluents, lubricants, binders, fillers, drugs, disintegrants or other additives. Thus, the dried or solid form may be comprised in a powder, granule, a pill, tablet or capsule.

In some embodiments, a dry or solid preparation of the disclosure exposed to water may result in a solution comprising (1) a bile acid, its derivative, its salt, or its conjugate with an amine, (2) water, and (3) a sufficient quantity of an aqueous soluble starch conversion product such that the bile acid and the starch conversion product remain in solution at any pH within a selected pH range. According to some embodiments, a dry or solid preparation of the disclosure exposed to water may result in a solution comprising (1) a bile acid, its derivative, its salt, or its conjugate with an amine, (2) water, and (3) a sufficient quantity of an aqueous soluble non-starch polysaccharide and an aqueous soluble starch conversion product such that the bile acid and the polysaccharide remain in solution at any pH within a selected pH range.

Dry or solid forms of the disclosure exposed to water may result in solutions further comprising resistant maltodextrin, an aqueous soluble ginseng extract, a pharmaceutical compound in a pharmaceutically appropriate amount, an aqueous soluble bismuth compound, or combinations thereof. Where the solution comprises one or more such materials, the solution composition may be adjusted to ensure that these materials remain in solution.

### EXAMPLES

Some embodiments of the present disclosure may be understood in connection with the following examples. However, one skilled in the art will readily appreciate the specific materials, compositions, and results described are merely illustrative of the disclosure, and are not intended to, nor should be construed to, limit the scope disclosure and its various embodiments.

### Example 1

### Preparation of Bile Acid Solution—Formulation 60

A sodium hydroxide solution was prepared by dissolving 5.2 g of extra pure grade (EP) NaOH in 100 mL of USP pharmaceutical grade water. Next, 48 g of UDCA was added to make a clear solution A.

A clear solution B was prepared by completely dissolving 320 g of food grade (NF) maltodextrin and 320 g of food grade (NF) soluble resistant starch in 300 mL of USP pharmaceutical grade water.

Solution A and solution B were combined with agitation and then, adequate amount of food grade sodium bisulfite (0.3 g/kg) was added to this clear solution. Food grade diluted phosphoric acid was added to adjust pH of this final solution (pH: 6-7.5). If necessary, this final solution may be filtered and/or heated to sterilize at 80° C, to 100° C.

### Example 2

### Preparation of Bile Acid Solution—Formulation 25

A sodium hydroxide solution was prepared by dissolving 2.7 g of EP NaOH in 100 mL of USP pharmaceutical grade water. Next, 25 g of UDCA was added to make a clear solution A.

A clear solution B was prepared by completely dissolving 500 g of NF maltodextrin and 150 g of NF soluble resistant starch in 400 mL of USP pharmaceutical grade water.

Solution A and solution B were combined with agitation and then, adequate amount of food grade sodium bisulfite (0.3 g/kg) was added to this clear solution. Food grade diluted phosphoric acid was added to adjust pH of this final solution (pH: 6-7.5). If necessary, this final solution may be filtered and/or heated to sterilize at 80° C. to 100° C.

### Example 3

### Preparation of Bile Acid Solution—Formulation 20

A sodium hydroxide solution was prepared by dissolving 2.2 g of EP NaOH in 100 mL of USP pharmaceutical grade water. Next, 20 g of UDCA was added to make a clear solution A.

A clear solution B was prepared by completely dissolving 500 g of NF maltodextrin and 150 g of NF soluble resistant starch in 400 mL of USP pharmaceutical grade water.

Solution A and solution B were combined with agitation and then, adequate amount of food grade sodium bisulfite (0.3 g/kg) was added to this clear solution. Fodd grade diluted phosphoric acid was added to adjust pH of this final solution (pH: 6-7.5). If necessary, this final solution may be filtered and/or heated to sterilize at 80° C. to 100° C.

### Example 4

### Preparation of Bile Acid Solution—Formulation 15

A sodium hydroxide solution was prepared by dissolving 1.72 g of EP NaOH in 100 mL of USP pharmaceutical grade water. Next, 15 g of UDCA was added to make a clear solution A.

A clear solution B was prepared by completely dissolving 450 g of NF maltodextrin and 200 g of NF soluble resistant starch in 400 mL of USP pharmaceutical grade water.

Solution A and solution B were combined with agitation and then, adequate amount of food grade sodium bisulfite (0.3 g/kg) was added to this clear solution. Food grade diluted phosphoric acid was added to adjust pH of this final solution (pH: 6-7.5). If necessary, this final solution may be filtered and/or heated to sterilize at 80° C. to 100° C.

### Example 5

### Preparation of Bile Acid Solution—Formulation 10

A sodium hydroxide solution was prepared by dissolving 1.1 g of EP NaOH in 100 mL of USP pharmaceutical grade water. Next, 10 g of UDCA was added to make a clear solution A.

A clear solution B was prepared by completely dissolving 300 g of NF maltodextrin and 300 g of NF soluble resistant starch in 400 mL of USP pharmaceutical grade water.

Solution A and solution B were combined with agitation and then, adequate amount of food grade sodium bisulfite (0.3 g/kg) was added to this clear solution. Food grade diluted phosphoric acid was added to adjust pH of this final solution (pH: 6-7.5). If necessary, this final solution may be filtered and/or heated to sterilize at 80° C. to 100° C.

### Example 6

### Preparation of Bile Acid Solution—Formulation 5

A sodium hydroxide solution was prepared by dissolving 0.55 g of EP NaOH in 100 mL of USP pharmaceutical grade water. Next, 5 g of UDCA was added to make a clear solution A.

A clear solution B was prepared by completely dissolving 150 g of NF maltodextrin and 150 g of NF soluble resistant starch in 450 mL of USP pharmaceutical grade water.

Solution A and solution B were combined with agitation and then, adequate amount of food grade sodium bisulfite (0.3 g/kg) was added to this clear solution. Food grade diluted phosphoric acid was added to adjust pH of this final solution (pH: 6-7.5). If necessary, this final solution may be filtered and/or heated to sterilize at 80° C. to 100° C.

### Example 7

### Preparation of Jelly comprising Bile Acid

A sodium hydroxide solution was prepared by dissolving 1.72 g of extra pure grade (EP) NaOH in 100 mL of USP pharmaceutical grade water. Next, 15 g of UDCA was added to make a clear solution A.

A solution B was prepared by adding 450 g of food grade (NF) maltodextrin, 100 g of food grade hydroxyethylcellulose (thickening agent) and 100 g of NF soluble resistant starch in 300 mL of USP pharmaceutical grade water.

Solution A and solution B were combined with agitation and then, adequate amount of food grade sodium bisulfite (0.3 g/kg) was added to this solution. This resulting solution was heated in order to make completely clear solution and to sterilize at 80° C. to 100° C.

### Example 8

### Preparation of Acacia Mucilage comprising Bile Acid

A sodium hydroxide solution was prepared by dissolving 1.1 g of EP NaOH in 100 mL of USP pharmaceutical grade water. Next, 10 g of UDCA was added to make a clear solution A.

A solution B was prepared by adding 300 g of food grade (NF) maltodextrin and 200 g of food grade (NF) acacia (gum Arabic; thickening agent) in 400 mL of USP pharmaceutical grade water.

Solution A and solution B were combined with agitation and then, adequate amount of food grade sodium bisulfite (0.3 g/kg) was added to this solution. This resulting solution was heated in order to make completely clear solution and to sterilize at 80° C. to 100° C.

### Example 9

### Animal Experiments—Procedure

Four-week old male ICR mice, each initially weighing 23-25 g, divided into two experimental groups. One group of 10 mice received AOM alone and a second group of 10 mice received AOM and a bile composition of the disclosure.

Mice in the AOM group were given a single intraperitoneal injection of AOM (10 mg/kg body weight). Starting one week after AOM injection, mice in the AOM group gained free access to drinking water that contained 1% Dextran sulfate sodium salt (DSS: Tumor formation accelerator) for 7 days and then followed without any further treatment.

In the AOM+bile acid group, mice were orally given 0.1 ml of Formulation 15 (Example 4) every day for one week and then, were given a single intraperitoneal injection of AOM (10 mg/kg body weight). Starting one week after AOM injection, mice in the AOM+bile acid group gained free access to drinking water that contained 1% Detran sulfate sodium salt for 7 days. Mice in this group continued to be fed 0.1 mL of Formulation 15 every day until death.

### Example 10

### Animal Experiments—Results

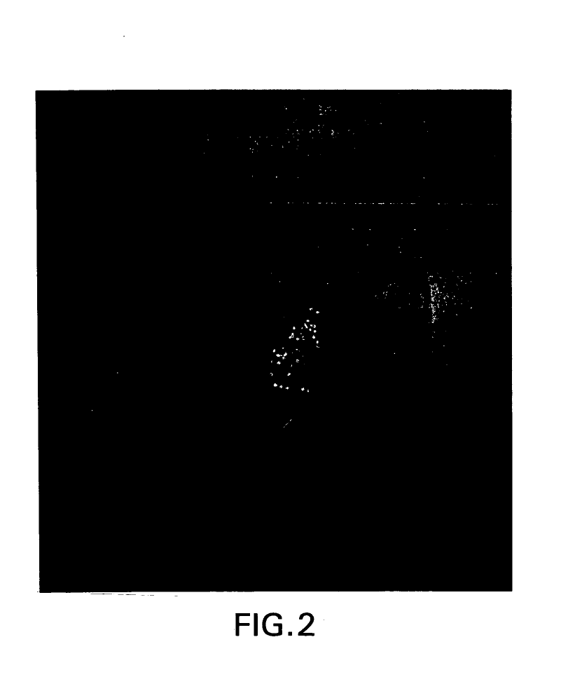

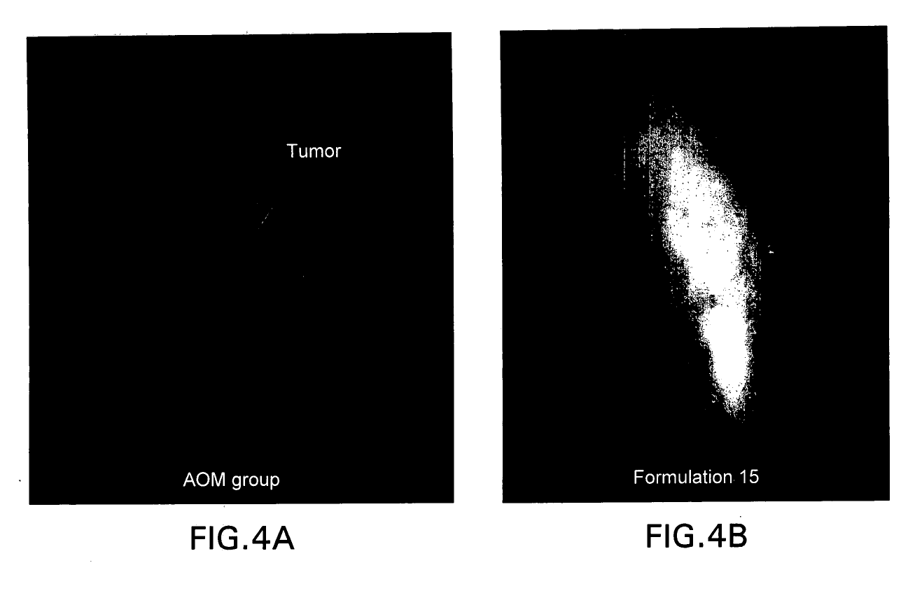

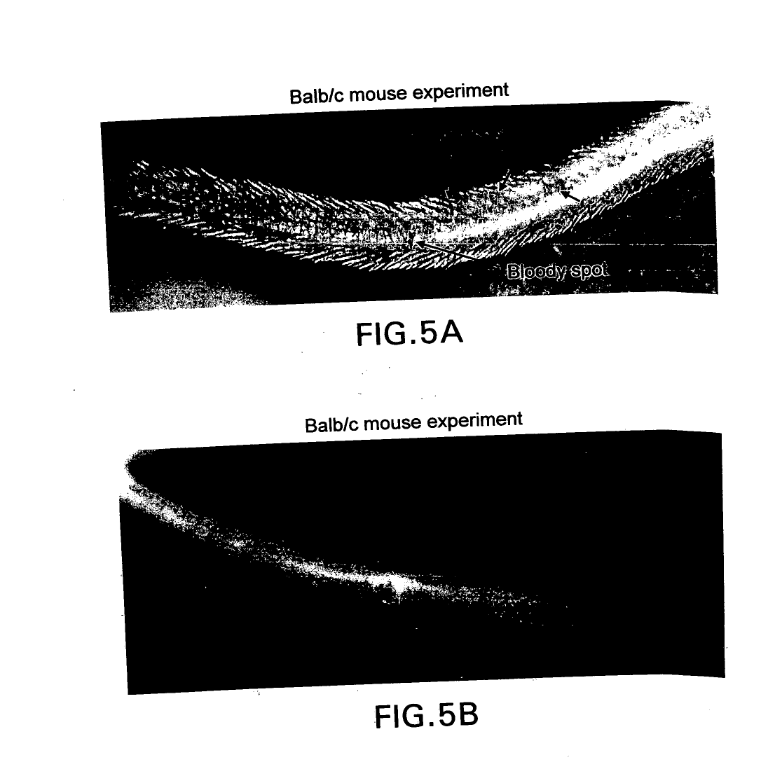

In general, mice in the AOM+bile acid group were much healthier than those in the AOM group by visual observation during experiments. All mice in the AOM group did not look healthy. All mice in the AOM group had one or more tumors on their back and head (FIG. 4A) and 5 mice in the AOM group were blind (FIG. 2). Abnormal behaviors such as no-standing, no-running and just rolling on the floor were observed from the mice having tumor on their back and head. But, this abnormal behavior was observed from only one mouse in the AOM+bile acid group.

Average mouse body weight in the AOM group and in the AOM+bile acid group increased up to 4 weeks and then, average mouse body weight in the AOM group was significantly reduced until death (FIG. 7). Average mouse body weight in the AOM+bile acid group after 4 weeks was kept substantially constant until death (FIG. 7).

While all mice tails in the AOM group had several bloody spots (FIG. 5A), mice tails in the AOM+bile acid group did not show any bloody spots (FIG. 5B).

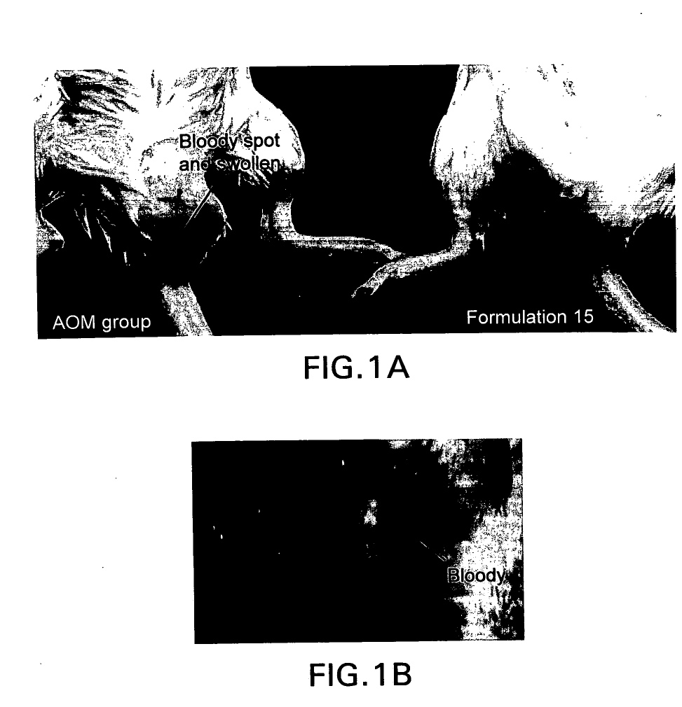

While all testes of mice in the AOM group swelled greatly and had several bloody spots, none of mouse testes in the AOM+bile acid group swelled or had bloody spots (FIGS. 1A and 1B).

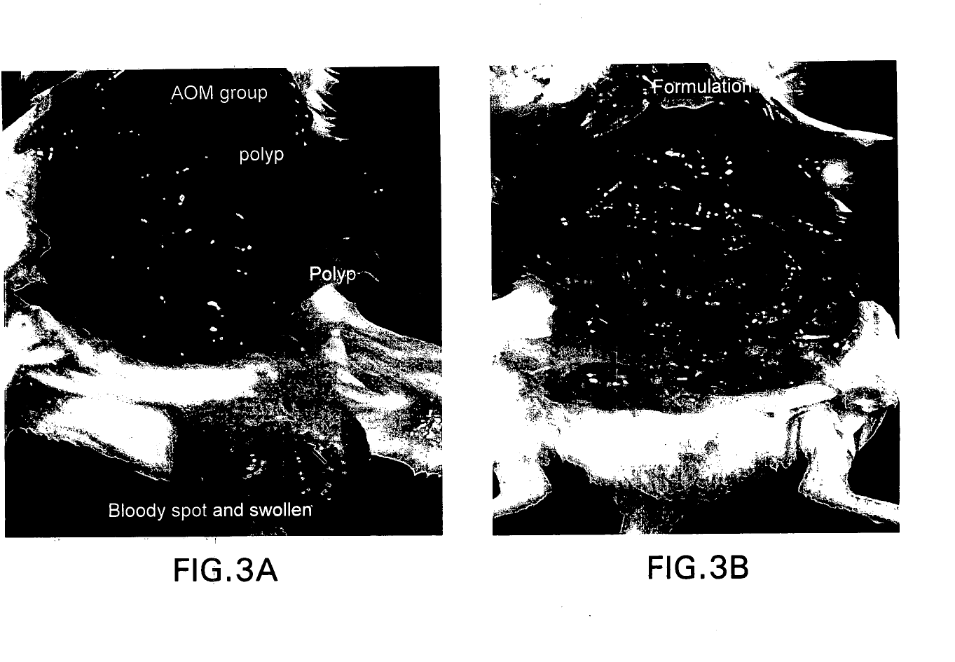

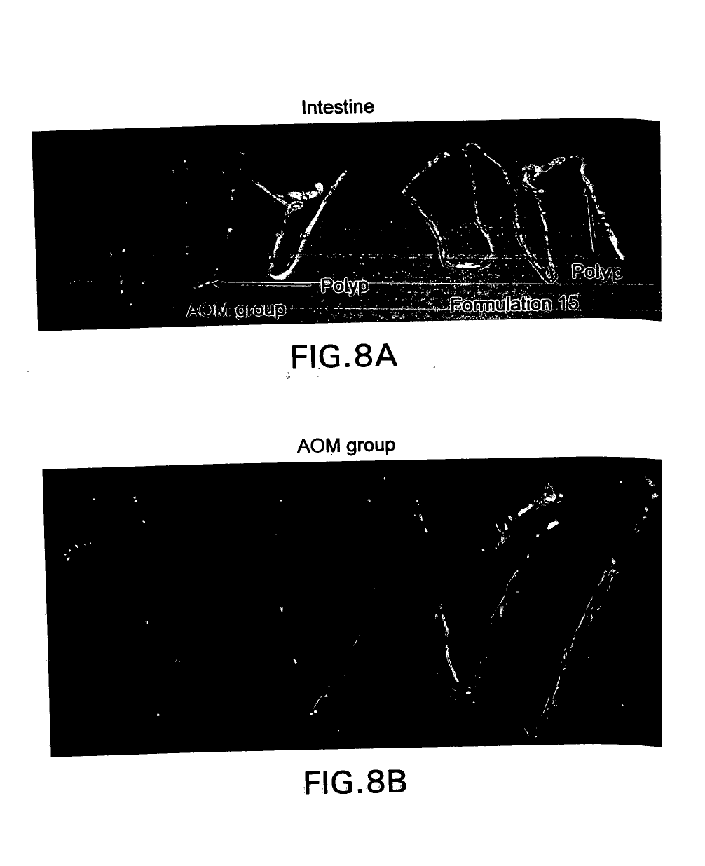

Several adenomatous polyps on entire colorectum were observed by visual inspection in all mice of the AOM-treated group (FIGS. 3A and 8A (left)), but few were observed in the AOM+bile acid group (FIGS. 3B and 8B (right)). Only one adenomatous polyp was observed from two mice in the AOM+bile acid group.

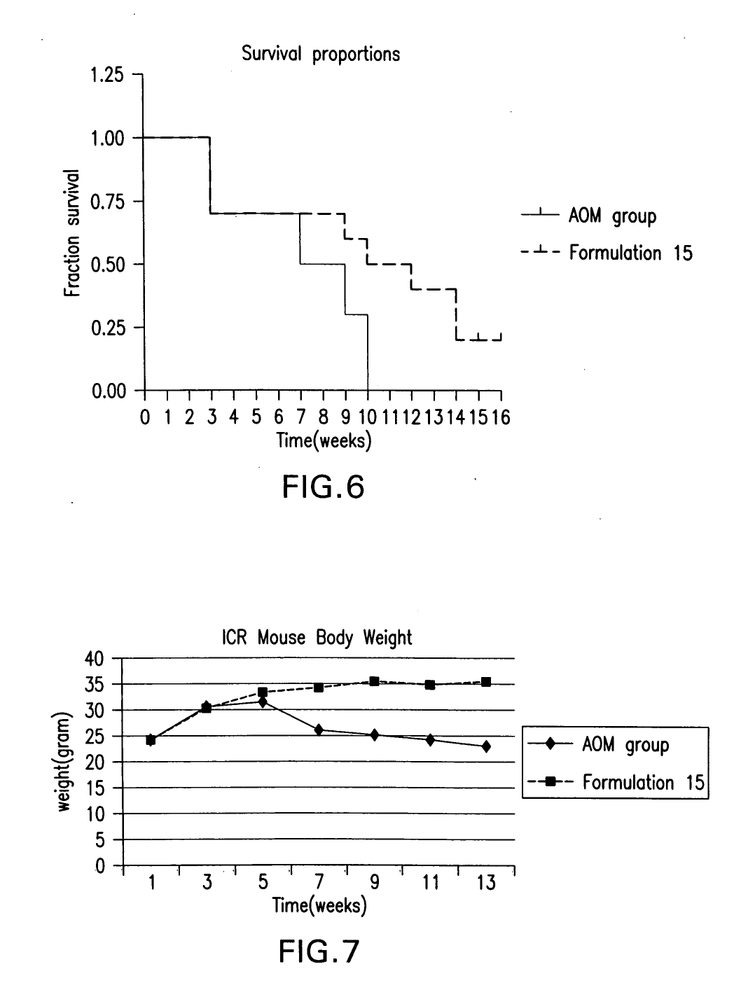

All mice in the AOM group died within 10 weeks of the day of AOM injection (FIG. 6). Of the mice in the AOM+bile acid group, 60% survived at 10 weeks and 40% survived at 14 weeks from the day of AOM injection (FIG. 6). Moreover, 20% of mice (2 mice) in the AOM+bile acid group survived for 16 weeks (FIG. 6).

Six mice in the AOM+bile acid group died within 3 weeks, possibly from shock. If these mice are excluded, the survival rates in this group are even higher: 86%, 57%, and 29% at 10, 14, and 16 weeks respectively.

What is claimed is:

1. A method of protecting a colorectum against adenomatous polyposis coli in a subject comprising:
administering to the subject a composition comprising:
(a) a first material selected from the group consisting of a bile acid, an aqueous soluble derivative of a bile acid, a bile acid salt, and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

2. A method of extending a life of a subject having adenomatous polyposis coli, said method comprising:
administering to the subject a composition comprising:
(a) a first material selected from the group consisting of a bile acid, an aqueous soluble derivative of a bile acid, a bile acid salt, and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

3. A method of maintaining a total body weight in a subject having adenomatous polyposis coli, said method comprising:
administering to the subject a composition comprising:
(a) a first material selected from the group consisting of a bile acid, an aqueous soluble derivative of a bile acid, a bile acid salt and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

4. A method of ameliorating or treating at least one symptom of adenomatous polyposis coli in a subject having or at risk of having adenomatous polyposis coli, said method comprising:
administering to the subject a composition comprising:
(a) a first material selected from the group consisting of a bile acid, an aqueous soluble derivative of a bile acid, a bile acid salt, and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

5. A method of reducing recurrence of colorectal adenomas in a colorectum of a subject comprising:
administering to the subject a composition comprising:
(a) a first material selected from the group consisting of a bile acid, an aqueous soluble derivative of a bile acid, a bile acid salt and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

6. A method of extending a life of a subject having colorectal cancer, said method comprising:
administering to the subject a composition comprising:
(a) a first material selected from the group consisting of a bile acid, an aqueous soluble derivative of a bile acid, a bile acid salt and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

7. A method of maintaining a total body weight in a subject having colorectal cancer, said method comprising:
administering to the subject a composition comprising:
(a) a first material selected from the group consisting of a bile acid, an aqueous soluble derivative of a bile acid, a bile acid salt and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

8. A method of ameliorating or treating at least one symptom of colorectal cancer in a subject having or at risk of having colorectal cancer, said method comprising:
administering to the subject a composition comprising:
(a) a first material selected from the group consisting of a bile acid, an aqueous soluble derivative of a bile acid, a bile acid salt and 7-ketolithocholic acid;
(b) a carbohydrate selected from the group consisting of an aqueous soluble starch conversion product, an aqueous soluble dietary carbohydrate that escape digestion and absorption in the small intestine, and combinations thereof; and
(c) water,
wherein the first material and the carbohydrate both remain in solution for all pH values of the solution within a selected range of pH values.

9. A method according to claim 1, wherein the composition further comprises a pharmaceutical in a pharmaceutically effective amount.

10. A method according to claim 8, wherein the composition further comprises a pharmaceutical in a pharmaceutically effective amount.

11. A method according to claim 9, wherein the pharmaceutical is selected from the group consisting of aspirin, methyl salicylate, diflunisal, indomethacin, sulindac, diclofenac, ibuprofen, ketoprofen, naproxen, ketorolac, mefenamic acid, piroxicam, meloxicam, coxibscelecoxib rofecoxib, valdecoxib, parecoxib, etoricoxib, nimesulide. oxaliplatin, leucovorin, irinotecan, cimetidine, salicylic acid, 2, 2-Bis (4-(4-amino-3-hydroxyphenoxy) phenyl) adamantane (DPA), paclitaxel, oxaliplatin, 5-fluorouracil, azathioprine, mycophenolate mofetil, cyclosporine, mycophenolic acid, tacrolimus, sirolimus, basiliximab, daclizumab, anti-thymocyte globulin (Rabbit), allopurinol, palonosetron, dolasetron, pamidronate, rasburicase, aprepitant, amifostine, gefitinib, palifermin, granisetron, sargramostim, levothyroxine, dronabinol, pegfilgrastim, interleukin eleven, filgrastim, octreotide, cinacalcet, levothyroxine, Liotrix, dexrazoxane, ondansetron, zoledronic acid, celecoxib, fenoprofen, benorylate, faislamine, amoxiprin, carprofen, flurbiprofen, loxoprofen, tiaprofenic acid, meclofenamic, ketorolac, oxaprozin, etodolac, nabumetone, mesalamine, balsalazide, bevacizumab, alemtuzumab, cetuximab, aldesleukin, ibritumomab tiuxetan, pemetrexed, tositumomab, gemcitabine, imatinib, trastuzumab, altretamine, topotecan, interferon alfa-2b, procarbazine, gemtuzumab ozogamicin, vinorelbine, mitoxantrone, denileukin diftitox, rituximab, erlotinib, bexarotene, arsenic trioxide, bortezomib, tretinoin, doxorubicin, dactinomycin, epirubicin, idarubicin, pentostatin, busulfan, temozolomide, melphalan, chlorambucil, mechlorethamine HC, clofarabine, cytarabine, cladribine, mercaptopurine, thioguanine, capecitabine, bicalutamide, flutamide, anastrozole, exemestane, Fulvestrant, letrozole, estramustine , leuprolide, triptorelin pamoate, histrelin, goserelin, porfimer, rotenone, thenoyltrifluoroacetone (TTFA), antimycin A, myxothiazol or oligomycin dexamethasone, methylprednisolone, hydrocortisone, prednisolone, rotenone, thenoyltrifluoroacetone (TTFA), antimycin A, myxothiazol, oligomycin, valdecoxib, rofecoxib, parecoxib and etoricoxib, atorvastatin, cerivastatin, fluvastatin, lovastatin, pravastatin, simvastatin, rosuvastatin, mevastatin, pitavastatin.

12. A method according to claim 10, wherein the pharmaceutical is selected from the group consisting of aspirin, methyl salicylate, diflunisal, indomethacin, sulindac, diclofenac, ibuprofen, ketoprofen, naproxen, ketorolac, mefenamic acid, piroxicam, meloxicam, coxibscelecoxib rofecoxib, valdecoxib, parecoxib, etoricoxib, nimesulide. oxaliplatin, leucovorin, irinotecan, cimetidine, salicylic acid, 2, 2-Bis (4-(4-amino-3-hydroxyphenoxy) phenyl) adamantane (DPA), paclitaxel, oxaliplatin, 5-fluorouracil, azathioprine, mycophenolate mofetil, cyclosporine, mycophenolic acid, tacrolimus, sirolimus, basiliximab, daclizumab, anti-thymocyte globulin (Rabbit), allopurinol, palonosetron, dolasetron, pamidronate, rasburicase, aprepitant, amifostine, gefitinib, palifermin, granisetron, sargramostim, levothyroxine, dronabinol, pegfilgrastim, interleukin eleven, filgrastim, octreotide, cinacalcet, levothyroxine, Liotrix, dexrazoxane, ondansetron, zoledronic acid, celecoxib, fenoprofen, benorylate, faislamine, amoxiprin, carprofen, flurbiprofen, loxoprofen, tiaprofenic acid, meclofenamic, ketorolac, oxaprozin, etodolac, nabumetone, mesalamine, balsalazide, bevacizumab, alemtuzumab, cetuximab, aldesleukin, ibritumomab tiuxetan, pemetrexed, tositumomab, gemcitabine, imatinib, trastuzumab, altretamine, topotecan, interferon alfa-2b, procarbazine, gemtuzumab ozogamicin, vinorelbine, mitoxantrone, denileukin diftitox, rituximab, erlotinib, bexarotene, arsenic trioxide, bortezomib, tretinoin, doxorubicin, dactinomycin, epirubicin, idarubicin, pentostatin, busulfan, temozolomide, melphalan, chlorambucil, mechlorethamine HC, clofarabine, cytarabine, cladribine, mercaptopurine, thioguanine, capecitabine, bicalutamide, flutamide, anastrozole, exemestane, Fulvestrant, letrozole, estramustine, leuprolide, triptorelin pamoate, histrelin, goserelin, porfimer, rotenone, thenoyltrifluoroacetone (TTFA), antimycin A, myxothiazol or oligomycin dexamethasone, methylprednisolone, hydrocortisone, prednisolone, rotenone, thenoyltrifluoroacetone (TTFA), antimycin A, myxothiazol, oligomycin, valdecoxib, rofecoxib, parecoxib and etoricoxib, atorvastatin, cerivastatin, fluvastatin, lovastatin, pravastatin, simvastatin, rosuvastatin, mevastatin, pitavastatin.

13. A method according to claim 8, wherein the composition is comprised in an enema.

14. A method according to claim 8, wherein the composition is comprised in a jelly.

15. A method according to claim 14, wherein the jelly further comprises a thickening agent selected from the group consisting of a water soluble polysaccharide and a synthetic cellulose derivative which swells in water.

16. A method according to claim 14, wherein the mucilage further comprises a thickening agent selected from the group consisting of acacia, chondrus, gelatin, xanthan gum carboxymethylcellulose, hydroxyethylcellulose, hydroxypropylcellulose, and hydroxypropylmethylcellulose.

17. A method according to claim 16, wherein the thickening agent is hydroxyethylcellulose.

18. A method according to claim 8, wherein the composition is comprised in a mucilage.

19. A method according to claim 18, wherein the mucilage further comprises a thickening agent selected from the group consisting of a water soluble polysaccharide and a synthetic cellulose derivative which swells in water.

20. A method according to claim 18, wherein the mucilage further comprises a thickening agent selected from the group consisting of acacia, chondrus, gelatin, xanthan gum carboxymethylcellulose, hydroxyethylcellulose, hydroxypropylcellulose, and hydroxypropylmethylcellulose.

21. A method according to claim 20, wherein the thickening agent is acacia.

22. A method according to claim 8, wherein a composition is comprised in a solution dosage form.

23. A method according to claim 8, wherein the composition is comprised in a suppository.

24. A method according to claim 8, wherein the subject is a mammal.

25. A method according to claim 8, wherein the subject is a human.

26. A method according to claim 8, wherein the first material is present in therapeutically effective amount.

27. A method according to claim 8, wherein the first material is selected from the group consisting of ursodeoxycholic acid, hyodeoxycholic acid, 7-ketolithocholic acid and sodium salt of ursodeoxycholic acid.

28. A method according to claim 8, wherein the first material is ursodeoxycholic acid or sodium salt of ursodeoxycholic acid.

29. A method according to claim 8, wherein the aqueous soluble starch conversion product is selected from the group consisting of maltodextrin, dextrin, liquid glucose, corn syrup solid, and soluble starch.

30. A method according to claim 8, wherein the aqueous soluble starch conversion product is maltodextrin.

31. A method according to claim 8, wherein the aqueous soluble starch conversion product is corn syrup solid.

32. A method according to claim 8, wherein the aqueous soluble dietary carbohydrate that escapes digestion and absorption in the small intestine is selected from the group consisting of non-digestible oligosaccharides, resistant starch, and non-starch polysaccharides

33. A method according to claim 32, wherein the soluble dietary carbohydrates that escape digestion and absorption in the small intestine is soluble non-starch polysaccharide

34. A method according to claim 8, wherein the soluble dietary carbohydrate that escapes digestion and absorption in the small intestine is selected from the group consisting of digestion resistant maltodextrin (fiber sol-2), guar gum, pectin, locust bean gum, cellulose, b-glucan and psyllium fibres

35. A method according to claim 34, wherein the soluble dietary carbohydrate that escapes digestion and absorption in the small intestine is an aqueous soluble digestion resistant maltodextrin (fibersol-2).

36. A method according to claim 8, wherein the weight ratio of the aqueous soluble starch conversion product to the aqueous soluble dietary carbohydrate that escapes digestion and absorption in the small intestine is 1-99:99-1.

37. A method according to claim 8, wherein the selected pH range is between about 1 and about 10.

38. A clear aqueous solution comprising:
(a) a first material selected from the group consisting of a bile acid, an aqueous soluble derivative of a bile acid, a bile acid salt, and 7-ketolithocholic acid;
(b) a carbohydrate selected from the group consisting of an aqueous soluble starch conversion product, an aqueous soluble digestion resistant maltodextrin (fibersol-2), and combinations thereof; and
(c) water,
wherein the first material and the carbohydrate both remain in solution for all pH values of the solution within a selected range of pH values.

39. A method according to claim 38, wherein the weight ratio of the aqueous soluble starch conversion product to the aqueous soluble digestion resistant maltodextrin (fibersol-2) is 1-99:99-1.

40. An aqueous solution comprising:
(a) a first material selected from the group consisting of a bile acid, an aqueous soluble derivative of a bile acid, a bile acid salt, and a bile acid conjugated with an amine by an amide linkage;
(b) a second material comprising a high molecular weight aqueous soluble starch conversion product; and
(c) water,
wherein the first and second materials both remain in solution for all pH values of the solution within a selected range of pH values.

41. A composition according to claim 40 further comprising an alkali.

42. A composition according to claim 41, wherein the alkali is selected from the group consisting of ammonia, sodium hydroxide, sodium carbonate, potassium hydroxide, potassium carbonate, calcium hydroxide, and calcium carbonate.

43. A composition according to claim 41, wherein the molar ratio of base to bile acid is from about 1.0 to about 1.3.

44. A method of protecting a colorectum against adenomatous polyposis coli in a subject comprising:
administering to the subject a dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and, combinations thereof;
(b) a second material selected from the group consisting of an aqueous soluble starch conversion product, a resistant maltodextrin, an aqueous soluble non-starch polysaccharide, and combinations thereof; and,
(c) a third material selected from aqueous soluble ginseng extract, aqueous soluble red ginseng extract, and combinations thereof.

45. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and combinations thereof; and
(b) an aqueous soluble starch conversion product;
wherein the first material and the aqueous soluble starch conversion product both remain in solution for all pH values of the solution within a selected range of pH values.
